(19) United States
(12) Patent Application Publication
(10) Pub. No.: US 2026/0248643 A1
(43) Pub. Date: Aug. 27, 2026

(54) External Footwear-Worn Heating Device

(71) Applicant: Jacob McNulty, Pittsfield, IL (US)

(72) Inventor: Jacob McNulty, Pittsfield, IL (US)

(21) Appl. No.: 19/281,889

(22) Filed: Jul. 28, 2025

Related U.S. Application Data
(60) Provisional application No. 63/763,502, filed on Feb. 26, 2025.

(51) Int. Cl. A61F 7/00; H05B 1/02; H05B 3/34

(52) U.S. Cl. CPC A61F 7/007; H05B 1/0272; H05B 3/342; A61F 2007/008

(57) ABSTRACT

An external footwear-worn heating device is provided. The device is designed to provide efficient toe-warming for users wearing steel-toe boots or other footwear in cold environments. The device comprises a lightweight, thermally conductive body that encloses a toe box of a boot and integrates at least one flexible heating element embedded within an insulated shell, ensuring even heat distribution and protection against external temperature fluctuations. A rechargeable battery powers the heating element and includes a waterproof charging port, a shock-resistant cover, and an optional removable design for extended use. The device features an adjustable heat control button with multiple settings, a power indicator for monitoring battery status, and a built-in temperature sensor to prevent overheating while optimizing energy consumption. The device is secured using an adjustable strap with an attachment mechanism, allowing quick and easy installation or removal.

### CROSS-REFERENCE TO RELATED APPLICATION

The present application claims priority to, and the benefit of, U.S. Provisional Application No. 63/763,502, which was filed on February 26, 2025, and is incorporated herein by reference in its entirety.

### FIELD OF THE INVENTION

The present invention relates generally to the field of foot-warming devices. More specifically, the present invention relates to an external footwear-worn heating device designed to provide efficient toe-warming for users wearing steel-toe boots or other footwear in cold environments. Accordingly, the present disclosure makes specific reference thereto. Nonetheless, it is to be appreciated that aspects of the present invention are also equally applicable to other like applications, devices, and methods of manufacture.

### BACKGROUND

Individuals who work in cold environments, particularly those who wear steel-toe boots, often face the challenge of keeping their feet warm. Steel-toe boots, while providing essential protection, contribute to heat loss because the metal toe cap conducts cold temperatures and lacks insulation. This issue is particularly severe for hunters, outdoorsmen, outdoor workers, construction professionals, and individuals working in unheated warehouses or in extreme weather conditions. Prolonged exposure to cold temperatures can cause discomfort, reduced mobility, and even serious health risks such as frostbite. Traditional solutions, such as thick socks or heated insoles, often fail to provide adequate warmth due to limitations in heat retention and distribution. Heated insoles, for example, may not fit comfortably within steel-toe boots and can interfere with proper foot support. Moreover, many existing warming solutions require direct contact with the foot, which can be impractical for individuals who need easily removable or adjustable heating options. Given these limitations, there is a need for an efficient, durable, and easily adjustable external heating solution specifically designed for steel-toe boots.

Therefore, there exists a long-felt need in the art for an external footwear-worn heating device that provides effective heat distribution without interfering with the interior comfort or fit of the footwear. There also exists a long-felt need in the art for an external footwear-worn heating device that offers various adjustable heat settings to accommodate different temperature conditions and user preferences. Moreover, there exists a long-felt need in the art for an external footwear-worn heating device that is durable, weather-resistant, and capable of maintaining consistent thermal performance in harsh working environments.

The subject matter disclosed and claimed herein, in one embodiment thereof, comprises an external footwear-worn heating device. The device is comprised of an external footwear-worn heating device designed to provide efficient toe-warming for users wearing steel-toe boots or other footwear in cold environments. The device comprises a lightweight, thermally conductive body that encloses a toe box of a boot and integrates at least one flexible heating element embedded within an insulated shell, ensuring even heat distribution and protection against external temperature fluctuations. A rechargeable battery powers the heating element and includes a waterproof charging port, a shock-resistant cover, and an optional removable design for extended use. The device features an adjustable heat control button with multiple settings, a power indicator for monitoring battery status, and a built-in temperature sensor to prevent overheating while optimizing energy consumption. An automatic shut-off mechanism enhances safety by turning off the device after prolonged inactivity. The device is secured using an adjustable strap with an attachment mechanism, allowing quick and easy installation or removal. As a result, the device ensures direct warmth to exposed foot areas without interfering with footwear fit or comfort, making it an effective and adaptable option for users in harsh working conditions.

In this manner, the external footwear-worn heating device of the present invention accomplishes all the foregoing objectives and provides a lightweight, efficient, and externally mounted heating solution for steel-toe boots and other footwear. More specifically, the thermally conductive body encloses the toe area while incorporating at least one heating element, ensuring even heat distribution without adding bulk to the boot interior. The inclusion of multiple adjustable heat settings allows users to customize warmth levels based on environmental conditions, improving comfort and usability. The device is also designed for easy attachment and removal using adjustable straps, allowing for quick application without modifying the boot structure. By securing the heat source externally, the device ensures direct warmth to the most exposed foot area while maintaining durability and adaptability for various footwear styles.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an external footwear-worn heating device. The external footwear-worn heating device provides toe-warming functionality for steel-toe boots and other footwear. The device comprises a body that houses the heating components, preferably made from carbon fiber or another lightweight, thermally conductive material. The body encloses the toe box of a boot and may extend to additional areas such as the instep, forefoot, or sides for increased heat coverage.

At least one heating element is integrated within the body, comprised of a flexible heating pad embedded in an insulated shell. The carbon fiber composition of the heating pad ensures efficient and even heat distribution, while the insulated shell retains heat and provides protection against external conditions. A rechargeable battery powers the heating element and includes at least one charging port. The battery may be housed in a shock-resistant cover for added durability and may also be removable to allow for extended use with interchangeable batteries.

The device features at least one button for adjusting multiple heat levels, such as low, medium, and high. A power indicator, such as an LED display or multicolor light, provides real-time feedback on battery status and heat settings. The heating element also includes a temperature sensor that regulates heat output to prevent overheating and optimize power usage.

The device is secured to a boot using at least one adjustable strap with an attachment mechanism. This ensures a secure fit while allowing for easy attachment and removal, while the external placement of the device enables direct heat application to the most exposed area of the foot without affecting the interior comfort of the footwear. Further, the lightweight construction of the device minimizes bulk, preserving natural movement for users in cold or harsh environments.

A method of using the device includes positioning the device over the toe box of a boot, securing the device with the adjustable strap, activating the heating element via the button, selecting the desired heat level, and using the power indicator for monitoring. The device can further be turned off and removed when heat is no longer needed.

Accordingly, the external footwear-worn heating device of the present invention is particularly advantageous as it provides a lightweight, efficient, and externally mounted heating solution for steel-toe boots and other footwear. More specifically, the thermally conductive body encloses the toe area while incorporating at least one heating element, ensuring even heat distribution without adding bulk to the boot interior. The inclusion of multiple adjustable heat settings allows users to customize warmth levels based on environmental conditions, improving comfort and usability. The device is also designed for easy attachment and removal using adjustable straps, allowing for quick application without modifying the boot structure. By securing the heat source externally, the device ensures direct warmth to the most exposed foot area while maintaining durability and adaptability for various footwear styles. In this manner, the external footwear-worn heating device overcomes the limitations of existing foot-warming devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a rear perspective view of one potential embodiment of an external footwear-worn heating device of the present invention in accordance with the disclosed architecture;

FIG. 2 illustrates a side perspective view of one potential embodiment of an external footwear-worn heating device of the present invention while attached to a boot in accordance with the disclosed architecture; and

FIG. 3 illustrates a flowchart of a method of using one potential embodiment of an external footwear-worn heating device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an external footwear-worn heating device that provides effective heat distribution without interfering with the interior comfort or fit of the footwear. There also exists a long-felt need in the art for an external footwear-worn heating device that offers various adjustable heat settings to accommodate different temperature conditions and user preferences. Moreover, there exists a long-felt need in the art for an external footwear-worn heating device that is durable, weather-resistant, and capable of maintaining consistent thermal performance in harsh working environments.

The present invention, in one exemplary embodiment, is comprised of an external footwear-worn heating device. The external footwear-worn heating device provides toe-warming functionality for steel-toe boots and other footwear. It comprises a body that houses the heating components, preferably constructed from carbon fiber or another lightweight, thermally conductive material. The body encloses the toe box of a boot and may extend to additional areas such as the instep, forefoot, or sides to enhance heat coverage.

At least one heating element is integrated within the body, consisting of a flexible heating pad embedded in an insulated shell. The carbon fiber composition of the heating pad ensures efficient and even heat distribution, while the insulated shell retains heat and protects against external conditions. A rechargeable battery powers the heating element and includes at least one charging port. The battery may be enclosed in a shock-resistant cover for added durability and may also be removable to allow for extended use with interchangeable batteries.

The device incorporates at least one button for adjusting multiple heat levels, such as low, medium, and high. A power indicator, such as an LED display or multicolor light, provides real-time feedback on battery status and heat settings. Additionally, the heating element includes a temperature sensor that regulates heat output to prevent overheating and optimize power usage.

The device is secured to a boot using at least one adjustable strap with an attachment mechanism. This ensures a secure fit while allowing for quick attachment and removal. The external placement of the device enables direct heat application to the most exposed area of the foot without affecting interior comfort. Furthermore, the lightweight construction minimizes bulk, preserving natural movement for users in cold or harsh environments.

A method of use includes positioning the device over the toe box of a boot, securing it with the adjustable strap, activating the heating element via the button, selecting the desired heat level, and using the power indicator for monitoring. The device can be turned off and removed when heat is no longer needed.

As a result, the external footwear-worn heating device offers a lightweight, efficient, and externally mounted heating solution for steel-toe boots and other footwear. The thermally conductive body encloses the toe area while incorporating at least one heating element, ensuring even heat distribution without adding bulk to the boot interior. Multiple adjustable heat settings allow users to customize warmth based on environmental conditions, enhancing comfort and usability. The device is designed for easy attachment and removal using adjustable straps, allowing for quick application without modifying the boot structure. By securing the heat source externally, the device provides direct warmth to the most exposed foot area while maintaining durability and adaptability for various footwear styles. In this manner, the external footwear-worn heating device overcomes the limitations of existing foot-warming solutions.

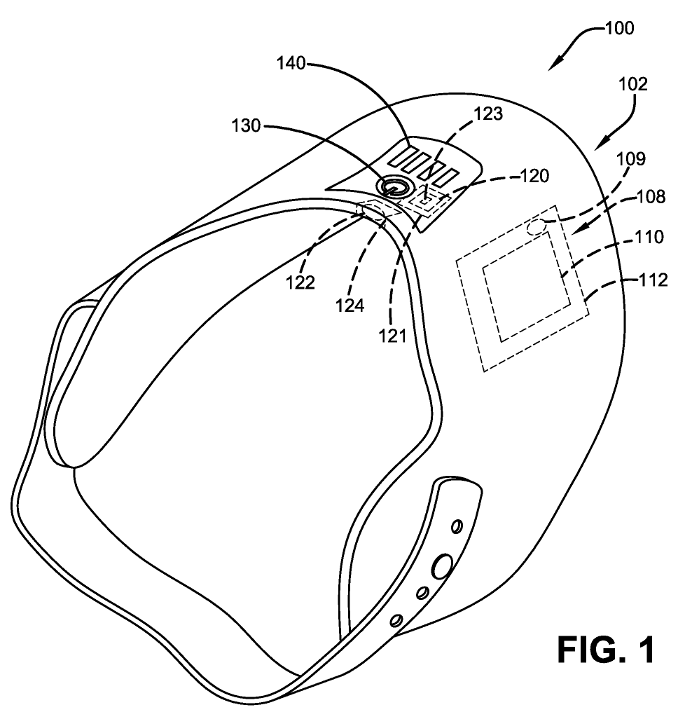

Referring initially to the drawings, FIG. 1 illustrates a rear perspective view of one potential embodiment of an external footwear-worn heating device 100 of the present invention in accordance with the disclosed architecture. The external footwear-worn heating device 100 provides an effective toe-warming solution that is worn on the outside of steel-toe boots 106 or other types of footwear. The device 100 is specifically designed to generate and maintain warmth in cold environments while remaining lightweight, durable, and adaptable to various boot styles and sizes.

The device 100 is comprised of a body 102 that serves as the primary housing for the heating components. The body 102 is preferably made from carbon fiber or another lightweight, thermally conductive material that enhances heat distribution while minimizing bulk. The body 102 preferably encloses the toe box 104 of a boot 106 or other footwear. In various embodiments, the body 102 may be configured to cover additional areas of the footwear, such as the instep, forefoot, or sides, to provide extended heat coverage as needed.

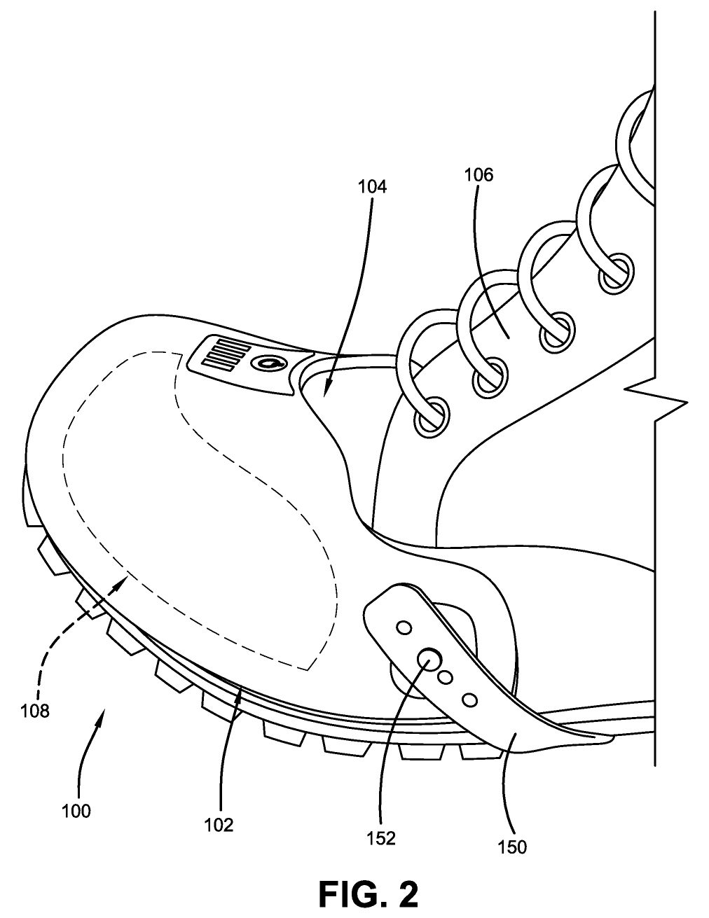

The body 102 incorporates at least one heating element 108 that generates warmth across the area covered by the body 102, as seen in FIG. 1 and FIG. 2. The heating element 108 is preferably at least one flexible heating pad 110 embedded within an insulated shell 112, which conforms to the shape of the toe box 104. The insulated shell 112 may be made from materials such as neoprene, thermoplastic polyurethane (TPU), silicone, or other heat-retaining and water-resistant materials. The carbon fiber composition of the heating pad 110 allows for efficient, even heat distribution, reducing the risk of hot spots while ensuring consistent thermal performance. The flexible insulated shell 112 surrounding the heating element 108 helps retain heat, protecting against external temperature fluctuations while maintaining comfort and adaptability to different boot designs.

A rechargeable battery 120 provides power to the heating element 108. The battery 120 may be comprised of at least one charging port 122, such as a USB-C, micro-USB, or a magnetic connector, enabling fast charging. To ensure protection against moisture, dust, and impact, the charging port 122 is covered by a waterproof cover 124, which may be made from rubber, silicone, or another flexible, durable material. The battery 120 may also be housed within a shock-resistant cover 121 made from reinforced silicone or rubber, further enhancing durability and ensuring reliable operation in wet or snowy conditions. In some variations, the battery 120 may be removable, allowing users to swap out depleted batteries 120 for charged batteries 120 for extended usage.

The device 100 features at least one button 130 that allows users to cycle through multiple adjustable heat levels, such as low, medium, and high, providing customizable warmth based on environmental conditions and personal comfort preferences. At least one power indicator 140, such as an LED display, multicolor light, or digital readout, provides real-time feedback on battery 120 status and heat settings, allowing users to monitor remaining power levels and make adjustments as needed.

To enhance safety and efficiency, the heating element 108 may be equipped with a built-in temperature sensor 109 that continuously monitors heat output to prevent overheating. The sensor 109 dynamically adjusts power delivery from the battery 120 to maintain consistent warmth while optimizing energy consumption. Additionally, the battery 120 may be comprised of an automatic shutoff mechanism 123 that powers down the device 100 after prolonged inactivity, preventing unnecessary power usage and further reducing the risk of overheating.

The device 100 is secured around a boot 106 (as seen in FIG. 2) using at least one adjustable strap 150. The strap 150 may be comprised of an attachment mechanism 152 such as, but not limited to, hook-and-loop fasteners, a quick-snap buckle, snap buttons, or an elastic tensioning system. These options allow for quick and easy attachment or removal while maintaining a snug fit that prevents the device 100 from shifting during use.

This device 100 is specifically designed to offer adjustable heat settings while maintaining durability, weather resistance, and ease of use. By securing externally over the toe portion of a user’s boots 106, the device 100 efficiently delivers warmth without interfering with the interior fit or comfort of the footwear. Unlike traditional insoles or socks, this external configuration ensures direct heat application to the most exposed area of the foot while also allowing for easy removal or replacement as needed. Further, the lightweight construction ensures that the device 100 does not add unnecessary bulk or weight to the footwear, preserving natural movement and comfort for users in cold or harsh working environments.

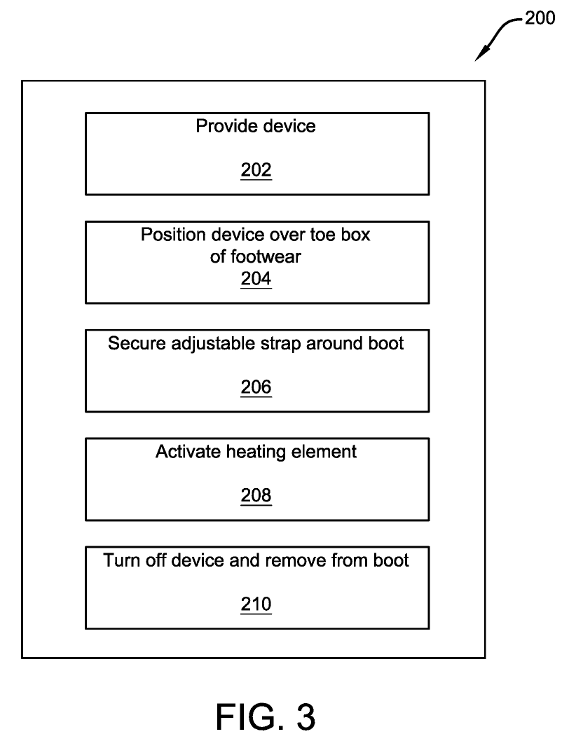

The present invention is also comprised of a method of using 200 the device 100, as seen in FIG. 3. First, a device 100 is provided, comprising a body 102, at least one heating element 108, at least one rechargeable battery 120, at least one adjustable strap 150, and at least one button 130 for adjusting heat levels [Step 202]. Then, the device 100 is positioned over the toe box 104 of a boot 106 or other footwear [Step 204]. Next, the adjustable strap 150 is secured around the boot 106 using an attachment mechanism 152 to ensure a secure fit [Step 206]. After securing the device 100, the user presses the button 130 to activate the heating element 108 and selects the desired heat level [Step 208]. The power indicator 140 provides real-time feedback on the battery 120 status and heat settings, allowing the user to monitor and adjust as needed. Finally, once the user no longer requires heat, the device 100 can be turned off by pressing the button 130, and it can be removed by releasing the adjustable strap 150 [Step 210].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein, “external footwear-worn heating device” and “device” are interchangeable and refer to the external footwear-worn heating device 100 of the present invention.

Notwithstanding the foregoing, the external footwear-worn heating device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the external footwear-worn heating device 100 as shown in the FIGS. are for illustrative purposes only, and that many other sizes and shapes of the external footwear-worn heating device 100 are well within the scope of the present disclosure. Although the dimensions of the external footwear-worn heating device 100 are important design parameters for user convenience, the external footwear-worn heating device 100 may be of any size, shape, and/or configuration that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An external footwear-worn heating device comprising:
a body configured to enclose a portion of a piece of footwear;
a heating element positioned within the body;
a rechargeable battery that provides power to the heating element; and
an adjustable strap attached to the body, the adjustable strap comprising an attachment mechanism for securing the body to the piece of footwear.

2. The external footwear-worn heating device of claim 1, wherein the heating element is comprised of a flexible heating pad.

3. The external footwear-worn heating device of claim 1, wherein the body is comprised of a carbon fiber material.

4. The external footwear-worn heating device of claim 1, wherein the heating element has an adjustable heat level.

5. The external footwear-worn heating device of claim 4 further comprised of a button that selects the adjustable heat level.

6. The external footwear-worn heating device of claim 1 further comprising a power indicator.

7. The external footwear-worn heating device of claim 1 further comprising a temperature sensor.

8. The external footwear-worn heating device of claim 1, wherein the rechargeable battery is positioned within a shock-resistant cover.

9. The external footwear-worn heating device of claim 1 further comprised of an automatic shutoff mechanism.

10. The external footwear-worn heating device of claim 1, wherein the attachment mechanism is comprised of a hook-and-loop fastener, a quick-snap buckle, a snap button, or an elastic tensioning system.

11. An external footwear-worn heating device comprising:
a body configured to enclose a portion of a piece of footwear;
a heating element positioned within the body, the heating element comprised of an insulated shell;
a rechargeable battery that provides power to the heating element and is further comprised of a charging port; and
an adjustable strap attached to the body, the adjustable strap comprising an attachment mechanism for securing the body to the piece of footwear.

12. The external footwear-worn heating device of claim 11, wherein the heating element is comprised of a flexible heating pad.

13. The external footwear-worn heating device of claim 11 further comprised of a waterproof cover that covers the charging port.

14. The external footwear-worn heating device of claim 11, wherein the rechargeable battery is positioned within a shock-resistant cover.

15. The external footwear-worn heating device of claim 11 further comprised of an automatic shutoff mechanism.

16. The external footwear-worn heating device of claim 11, wherein the insulated shell is comprised of a flexible material.

17. The external footwear-worn heating device of claim 11, wherein the heating element has an adjustable heat level.

18. The external footwear-worn heating device of claim 17 further comprised of a button that selects the adjustable heat level.

19. The external footwear-worn heating device of claim 11, wherein the attachment mechanism is comprised of a hook-and-loop fastener, a quick-snap buckle, a snap button, or an elastic tensioning system.

20. A method of using an external footwear-worn heating device, the method comprising the following steps:
providing an external footwear-worn heating device comprised of a body, a heating element, a rechargeable battery, an adjustable strap, and a button for adjusting a heat level of the heating element;
positioning the body over a piece of footwear;
securing the adjustable strap around the piece of footwear;
pressing a button of the external footwear-worn heating device to activate the heating element and to select the heat level; and
turning off the external footwear-worn heating device by pressing the button and removing the adjustable strap from the piece of footwear.
